(19) United States
(12) Patent Application Publication
(10) Pub. No.: US 2024/0374634 A1
(43) Pub. Date: Nov. 14, 2024

(54) NUTRACEUTICAL OPHTHALMIC COMPOSITION FOR THE TREATMENT OF RETINAL PATHOLOGIES HAVING A NEOVASCULAR COMPONENT

(71) Applicant: Laboratoires THEA, Clermont-Ferrand (FR)

(72) Inventors: Celine OLMIERE, Vertaizon (FR); Fabrice MERCIER, Chamalieres (FR)

(21) Appl. No.: 17/309,695

(22) PCT Filed: Dec. 19, 2019
(86) PCT No.: PCT/EP2019/086174

§ 371 Date: Jun. 15, 2021

(30) Foreign Application Priority Data
Dec. 20, 2018 (EP) 18306772.7

(51) Int. Cl. A61K 33/34; A61P 27/02

(52) U.S. Cl. CPC A61K 33/34; A61P 27/02

(57) ABSTRACT

The invention relates to a nutraceutical ophthalmic composition comprising resveratrol, advantageously in an amount greater than 20 mg, for administration to a subject receiving an anti-VEGF treatment.

### CROSS-REFERENCE TO RELATED APPLICATIONS

This application is the U.S. National Phase under 35 U.S.C. § 371 of International Application PCT/EP2019/086174, filed Dec. 19, 2019, which claims the benefit of European Application No. 18306772.7, filed Dec. 20, 2018, the disclosures of which are incorporated by reference herein in their entireties.

### FIELD OF THE INVENTION

The invention relates to a nutraceutical ophthalmic composition comprising, notably, resveratrol, intended for administration to a subject receiving an anti-VEGF treatment, in particular within the scope of the management of a retinal pathology with a neovascular component.

Surprisingly, the application demonstrates that the joint administration of such a composition and an anti-VEGF agent produces a beneficial effect on the level of VEGF produced and offers promising prospects for the treatment of these eye diseases.

### PRIOR ART

Throughout Europe, the majority of blindness and severe visual loss are caused by age-related retinal diseases. Given the current demographic trends, it is expected that the prevalence and incidences of these diseases will increase. Significant pressure on European health systems will be one of the direct economic consequences.

To date, Age-related Macular Degeneration (AMD) affects approximately 34 million people in Europe. It is expected that the number of AMD patients will increase by almost 25%, in view of how the population is set to grow and age by 2050. In addition, the current and future situation is similar for diabetes-related eye diseases, including Diabetic Macular Oedema (DMO). More than 25% of diabetic patients are affected by Diabetic Eye Diseases (DED), representing almost 4 million people throughout Europe. As the prevalence of diabetes increases significantly owing to demographic trends and lifestyle changes, this type of disease will also increase.

More specifically, AMD is the leading cause of visual impairment among people aged over 50 years old. In all its forms, this disease affects about 8% of the French population, but its frequency increases significantly with age: it affects 1% of people aged 50 to 55, approximately 10% of people aged 65 to 75 years old, and from 25% to 30% of people aged over 75 years old. If only late forms of the disease, combined with a loss of central vision, are taken into account, these figures can be approximately halved.

The disease selectively affects the macular region, i.e., the central area of the retina, resulting in a progressive loss of central vision, namely, the field of vision useful for reading, recognising faces or driving, etc. It is the main cause of poor vision among elderly people.

The disease begins with an early, non-degenerative phase called age-related maculopathy (ARM or “early dry”). This phase is characterised by the accumulation of small, whitish deposits (or “soft drusen”) in and around the macula. These deposits are visible during a simple fundus examination. This phase is usually asymptomatic, but the patient may perceive distortions of straight lines (“metamorphopsias”) and blurred spots.

During the most advanced stages, symptoms varying depending on the severity of the disease appear:
reduced visual acuity, with the need for better lighting for reading or any precision work;
increasingly blurred central vision, alteration of colour perception, distortion of straight lines, which appear distorted and warped;
the appearance of a dark spot in the centre of the visual field called a scotoma, difficulty in recognising faces, visual hallucinations or a sudden decrease in visual acuity;
severe damage in the second eye varies greatly from subject to subject: it may occur rapidly, within one year, within 10 years, or never occur at all.

An ARM may remain stable throughout one's life. Nevertheless, in approximately half of cases and under the influence of several factors, ARM evolves into late degenerative forms:
an atrophic form that is a dry form, in which the macula atrophies with age and is gradually replaced by scar tissue; or
a wet form that is an exudative form in which small blood vessels develop under the macula. These neovessels bleed easily and are therefore responsible for exudates and haemorrhages of the fundus of the eye. If exudative AMD is present in one eye, there is a risk of developing it in the second eye.

These two late forms have approximately the equivalent impact. They cause irreversible damage to the macula and loss of central vision that affect only one eye or both eyes. Mixed forms may be observed.

From a mechanistic point of view, the wet form of AMD, called neovascular or exudative AMD, as well as diabetic retinopathy, macular oedema, retinal vein occlusion (RVO) or even strong myopia, result in a proliferation of new abnormal vessels under the retina. These fragile vessels allow serum to be diffused, which is responsible for a lifting of the retina, and/or blood causing the appearance of retinal haemorrhages. This form develops rapidly if left untreated, with a loss of vision within a few weeks or even a few days. This process can be slowed down by taking anti-VEGF type medication.

VEGF (Vascular Endothelial Growth Factor) is a growth factor of the vascular endothelium that plays a role in the processes of angiogenesis, i.e. the budding of pre-existing vessels, and vasculogenesis corresponding to the formation of new vessels from endothelial progenitors. VEGF, and more particularly the VEGF-A isoform, binds to the surface of receptors with tyrosine-kinase activity, notably VEGF-R1 and VEGF-R2, which play a key role in angiogenesis.

Thus, since 2006, certain eye diseases such as the wet form of AMD (or “wet AMD”) have been treated with VEGF inhibitors. Progressively, other neovascular diseases such as diabetic macular oedema (DMO or DME), retinal vein occlusion (RVO) or myopic choroidal neovascularisation (CNV or PM) are also treated by these treatments.

Anti-VEGF agents include particularly antibodies or antibody fragments directed against VEGF or recombinant proteins capable of binding to VEGF receptors. Examples include ranibizumab (for example, LUCENTIS® marketed by NOVARTIS PHARMA SAS or its biosimilars developed by SAMSUNG, INTAS or FORMYCON), bevacizumab (for example, AVASTIN® marketed by ROCHE), aflibercept (for example, EYLEA® marketed by BAYER SANTE), abicipar pegol (being developed by ALLERGAN), conbercept (for example, the product being developed by CHENGDU KANGHON BIOTECH INC), faricimab (for example, the product RG7716 being developed by ROCHE), or Brolucizumab (NOVARTIS).

These anti-VEGF agents are usually administered by intravitreal injection, i.e., an injection through the conjunctiva and sclera, directly into the eye cavity behind the lens, i.e., the vitreous cavity containing the vitreous humour.

It involves directly injecting medications into the eye. This treatment has several advantages, compared with other routes of administration, notably topically:
a reduction in the amount of the active ingredient;
a more effective action;
an effective targeting of the area to be treated;
a reduction in adverse effects or a reduction in systemic passage.

However, intravitreal injection remains a technical procedure, performed by specialised practitioners, and requiring special aseptic and antiseptic conditions. Moreover, this injection can be the cause of a certain number of rather problematic side effects: endophthalmitis, detachment of the retina, ocular hypertension/glaucoma, cataract, inflammation, subconjunctival haemorrhage, systemic toxicity, inflammation, appearance of floating bodies, etc. In addition, it is generally not recommended to perform injections into both eyes on the same day in order to avoid total visual loss in the event of a problem or side effect following the injection. Indeed, these injections are usually followed by a very painful period for patients, which may require them to spend several hours in complete darkness.

The dosage, in particular the dose and frequency of the administration, may notably depend on the active ingredient and also on the pathology to be treated, with an average of seven injections per year.

For example, the treatment of AMD with ranibizumab or bevacizumab starts via an induction phase with 1 injection per month for 3 consecutive months, followed by a maintenance phase during which the patients' visual acuity is checked once per month. If the patient has a loss of visual acuity of more than 5 letters on the “Early Treatment Diabetic Retinopathy Study” (ETDRS) scale or the equivalent of one line on the Snellen scale, the agent should be re-administered. The interval between 2 doses must not be less than 1 month.

In terms of DMO, monthly injections of ranibizumab or bevacizumab are recommended until visual acuity stabilises at three consecutive monthly assessments on treatment.

In terms of RVO, monthly injections of ranibizumab or bevacizumab are recommended until visual acuity stabilises. Failure to improve visual acuity after 3 consecutive monthly injections justifies discontinuation of treatment.

In the presence of aflibercept, the protocol is slightly different:
In terms of AMD, at the initiation of treatment and as with other anti-VEGF agents, aflibercept is injected once per month for 3 consecutive months, then one injection is performed every 2 months, with no follow-up visit between injections. After the first 12 months of the treatment, the interval between 2 injections may be extended depending on visual and anatomical results. In this case, the follow-up schedule must be determined by the doctor administering the treatment, and the follow-up visits may be more frequent than the scheduled injections.

In terms of RVO, the recommended dose is 2 mg of aflibercept, corresponding to 50 μL. After the first injection, treatment is administered monthly. The interval between 2 injections must not be less than 1 month. If no improvement in the visual and anatomical parameters is observed after the first three injections, further treatment is not recommended. Monthly treatment is continued until stable visual and anatomical results are achieved during three monthly assessments. The need for further treatment can then be reassessed. If necessary, the treatment can be continued with a gradual increase in the interval between injections in order to maintain the visual and anatomical response.

In parallel to these treatments using anti-VEGF agents administered by intravitreal injection, other techniques are used to destroy neo-vessels, notably photocoagulation (thermal destruction of abnormal vessels) and photodynamics. The latter technique involves the intravenous injection of a photosensitive product (for example, verteporfin) which becomes toxic under the effect of red light applied locally with a laser.

Prior to these heavy treatments, which are performed at advanced stages of the disease. studies were performed in an attempt to find earlier and “lighter” treatments, with the aim of preventing the onset and delaying the progress of the disease.

Thus, studies called AREDS 1 (Age-Related Eye Disease Study Research Group. A Randomized, Placebo-Controlled, Clinical Trial of High-Dose Supplementation With

Vitamins C and E. Beta Carotene, and Zinc for Age-Related Macular Degeneration and Vision Loss: AREDS Report No. 8 and 9. Arch Ophthalmol. 2001) and AREDS 2 (Lutein+zeaxanthin and omega-3 fatty acids for age-related macular degeneration: the Age-Related Eye Disease Study 2 (AREDS2) randomized clinical trial, JAMA. 2013 May 15;309 (19): 2005-15. doi: 10.1001/jama.2013.4997) were performed in order to demonstrate the beneficial effect of dietary supplements on eye diseases.

These studies have revealed that antioxidant-based dietary supplements are potentially beneficial in relation to eye diseases, notably age-related diseases, particularly AMD.

In practice, the AREDS 1 study recommends a daily intake of antioxidants such as trace elements and vitamins, as follows:

| | | |
|---|---|---|
| Zinc | 80 | mg |
| Vitamin C | 500 | mg |
| Vitamin E | 267 | mg |
| Copper | 2 | mg |
| Beta-carotene (=Vitamin A) | 15 | mg |

These supplements could reduce the risk of visual loss in patients with advanced AMD.

The AREDS 2 study was performed to highlight the effects of lutein, zeaxanthin (as a replacement for beta-carotene) and omega-3 fatty acid supplements. This study recommended eliminating vitamin A, and reported the benefit of an additional daily intake of the following:

| | | |
|---|---|---|
| Lutein | 10 | mg |
| Zeaxanthin | 2 | mg |
| Docosahexaenoic acid (DHA) | 350 | mg |
| Eicosapentaenoic acid (EPA) | 650 | mg |

Based on the results of the AREDS studies, therefore, it is recommended that dietary supplements be taken daily by people with a high risk of developing AMD, particularly in patients having an ARM in one or both eyes, as well as in patients with advanced AMD still only involving one eye. However, these studies did not highlight a positive effect of these formulations on disease progression in patients with advanced AMD or on vision loss.

The NAT 2 study (Merle et al., Invest Ophthalmol Vis Sci. 2014 Mar. 28;55 (3):2010-9. doi: 10.1167/iovs.14-13916), including 300 patients over three years, demonstrated a 68% reduction in the risks of developing neovascular AMD after a significant increase in DHA in cells. Thus, this study is the first to explore the potential of long-term oral DHA supplementation to prevent the development of AMD.

Finally, document WO 2016/151269 describes a dietary supplement combining vitamins, trace elements, carotenoids, omega-3 fatty acids and resveratrol, and its use for the treatment and/or prevention of eye diseases. In particular, this document reports that the combination of the different compounds listed has a differential and beneficial action on the expression and activity of VEGF or its receptors (VEGF-R).

However, there is a clear need to develop new solutions in order to improve the management of eye diseases, notably retinal diseases with a neovascular component, meeting in particular the following expectations: a spacing of intravitreal injections of anti-VEGF, a reduction in the side effects linked to them, a reduction in public health costs and an improvement in the quality of life of patients.

### SUMMARY OF THE INVENTION

Surprisingly, the Applicant has highlighted that the administration of a dietary supplement would make it possible to increase, prolong or even improve the effects of current anti-VEGF treatments, notably, treatments by intravitreal injection, in patients receiving such treatments.

According to its first aspect, the invention relates to a nutraceutical ophthalmic composition comprising resveratrol and the administration of such a composition to a subject receiving anti-VEGF treatment, in particular subjected to treatment with an anti-VEGF agent.

Within the scope of the invention, a “nutraceutical composition” is defined as a composition whose purpose is to supplement the normal diet and constitute a concentrated source of nutrients or other substances which have a nutritional or physiological effect, alone or in combination. Advantageously, it involves a dietary supplement.

Within the scope of the invention, a composition with properties that are beneficial to eye health is referred to as an “ophthalmic composition”. However, this does not exclude the possibility that such a composition may have a beneficial effect on other human or animal organs.

Resveratrol is a polyphenolic compound, derived from stilbene, of which there are two isomers, but the trans form is predominantly active. Throughout the rest of the description, the term “resveratrol” therefore may be used instead of trans-resveratrol.

Resveratrol is a polyphenol present in abundant quantities in certain fruits, notably in grapes, oil seeds and wine. There are now several commercial sources of resveratrol which may be used for preparing a composition implemented within the scope of the invention:
Vitis vinifera:
   grape skin
   grape seed
Polygonum Cuspidatum
produced by fermentation

Preferred sources include:
resveratrol source Vitis vinifera, grape skin: for example, a natural extract with a content of 5%;
synthetic resveratrol 99% (CAS Number: 501-36,-0).

Resveratrol is advantageously present in an amount (daily dose) greater than or equal to 1 mg, or even 2 mg, 5 mg, 10 mg, 20 mg, 30 mg, 50 mg, 60 mg or even 100 mg or 200 mg. According to a particular embodiment, its daily quantity is greater than or equal to 20 mg or even greater than 20 mg, greater than or equal to 30 mg or even greater than 30 mg, greater than or equal to 50 mg, greater than or equal to 60 mg, or even greater than or equal to 100 mg. Advantageously, it is less than or equal to 1 g, preferably less than or equal to 500 mg, particularly preferably less than or equal to 200 mg. According to a particular embodiment, a composition implemented within the scope of the invention comprises a daily dose of resveratrol equal to 50 mg, 60 mg, 70 mg, 80 mg, 90 mg, 100 mg, 150 mg or 200 mg.

According to an advantageous embodiment, the composition further comprises vitamins, trace elements, carotenoids, and omega-3 fatty acids. Even more advantageously, the ingredients vitamins, trace elements, omega-3 fatty acids and carotenoids, known for their antioxidant properties, are selected as recommended by the AREDS studies.

According to a particular embodiment, the composition comprises at least one vitamin, advantageously a combination of vitamins. Advantageously, embodiments comprise vitamin C and/or vitamin E.

Some embodiments comprise the following vitamins:
nicotinamide, niacinamide or vitamin B3 or PP;
pyridoxine, for example in the form of pyridoxine HCl, or vitamin B6;
riboflavin or vitamin B2;
thiamine, for example in the form of thiamine nitrate, or vitamin B1;
cyanocobalamin or vitamin B12;
folic acid or vitamin B9;
vitamin B5;
vitamin B8;
vitamin D.

Vitamin C, available in the form of ascorbic acid or calcium ascorbate, is advantageously present in an amount (daily dose) of between 50 mg and 500 mg, for example 240 mg or 120 mg.

Vitamin E or alpha-tocopherol, for example, available in the form of 67% oil, is advantageously present in an amount (daily dose) of between 10 mg and 500 mg, for example equal to 30 mg.

According to another embodiment, the following daily amounts, expressed in mg, can be considered for the following vitamins:
vitamin B3 or PP: 1 to 100, for example 18;
vitamin B6: 1 to 10, for example 2;
vitamin B2: 1 to 10, for example 1.6;
vitamin B1: 1 to 10, for example 1.4:
vitamin B12: 0.0005 to 0.01, for example 0.001;
vitamin B9: 0.1 to 1, for example 0.2.

According to a particular embodiment, the composition does not comprise vitamin A or beta-carotene.

According to a particular embodiment, the composition comprises at least one trace element, advantageously a combination of trace elements. Advantageously, it involves zinc (Zn) and/or copper (Cu). It may also be the following trace elements:
manganese, for example anhydrous manganese sulphate;
selenium, for example, sodium selenite;
magnesium, for example, magnesium oxide.

Zinc (Zn), available as zinc oxide or zinc sulphate (monohydrate), is advantageously present in an amount (daily dose) of between 5 mg and 100 mg, for example 12.5 mg.

Copper (Cu), available in the form of copper sulphate, copper monohydrate or anhydrous copper, is advantageously present in an amount (daily dose) of between 0.2 mg and 10 mg, for example, an amount equal to 1 mg.

According to another embodiment, the following daily amounts, expressed in mg, may be considered for the following trace elements:
manganese: between 1 mg and 10 mg, for example 1 mg;
selenium: between 0.01 mg and 0.1 mg, for example 0.025 mg; and
magnesium: between 1 mg and 50 mg, for example 10 mg.

According to a particular embodiment, the composition comprises at least one carotenoid, advantageously a combination of carotenoids. Advantageously, it involves lutein and/or zeaxanthin. It may also be the following carotenoids:
meso-zeaxanthin;
lycopene;
astaxanthin.

Lutein, available in the form of 20% lutein by weight, is advantageously present in an amount (daily dose) of between 2 mg and 50 mg, for example 10 mg or 20 mg.

Zeaxanthin, available in the form of 5% or 14% zeaxanthin by weight, is advantageously present in an amount (daily dose) of between 0.5 mg and 10 mg, for example equal to 2 mg or 4 mg.

According to a particular embodiment, the composition comprises at least one polyunsaturated fatty acid of the omega-3 type, advantageously a combination of omega-3 fatty acids. Advantageously, it involves eicosapentaenoic acid (EPA) and/or docosahexaenoic acid (DHA). It may also be the following omega-3 fatty acids:
alpha-linolenic acid (ALA);
docosapentaenoic acid (DPA).

According to a particular embodiment, the composition comprises at least one polyunsatured fatty acid of omega 3 type, advantageously a combination of omega 3 fatty acids.

The omega-3 fatty acids used in such a composition may be derived from fish oil rich in omega-3 fatty acids, or from vegetable oil, for example linseed oil. These fatty acids are advantageously present in an amount (daily dose) of between 100 mg and 1000 mg. More generally, fish oil may be present in a daily amount of between 400 mg and 1000 mg, or even between 800 mg and 1000 mg.

In this context, EPA is advantageously present in an amount (daily dose) of between 100 mg and 1000 mg, for example 172 mg.

In this context, DHA is advantageously present in an amount (daily dose) of between 100 mg and 1000 mg, for example equal to 366 mg.

According to particular embodiments, the composition used within the scope of the invention may contain other active ingredients such as:
one or more other polyphenols, such as epigallocatechin-3-gallate (EGCG);
glutathione, for example 1 mg to 10 mg per day, or even 2 mg per day;
anthocyanoside(s);
hydroxythyrosol.

The content of each of these ingredients is easily determined and adjusted by the person skilled in the art.

Thus, and according to one embodiment, the composition comprises or consists of the following ingredients:
vitamin C;
vitamin E;
potentially vitamin D;
zinc;
copper;
lutein;
zeaxanthin;
omega 3 fatty acids from fish oil, notably EPA and DHA;
resveratrol.

According to another embodiment, the composition comprises or consists of the following ingredients on a daily dose basis:
50 mg to 500 mg of vitamin C, advantageously 240 mg, more advantageously 120 mg;
10 mg to 500 mg of vitamin E, advantageously 30 mg;
5 mg to 100 mg of zinc, advantageously 12.5 mg;
0.2 mg to 10 mg of copper, advantageously 1 mg;
2 mg to 50 mg of lutein, advantageously 20 mg;
0.5 mg to 10 mg of zeaxanthin, advantageously 4 mg;
400 mg to 1000 mg of fish oil, particularly advantageously 800 to 1000 mg;
resveratrol, advantageously in an amount greater than or equal to 20 mg, preferably greater than 30 mg, particularly preferably greater than 50 mg, very advantageously in an amount greater than or equal to 60 mg;
optionally vitamin D, advantageously 5 μg.

According to a preferred embodiment, the composition comprises or consists of the following ingredients, on a daily dose basis:
120 mg of vitamin C;
30 mg of vitamin E;
12.5 mg of zinc;
1 mg of copper;
20 mg oflutein;
4 mg of zeaxanthin;
950 mg of fish oil, advantageously containing 366 mg of DHA and 172 mg of EPA;
resveratrol, advantageously in an amount greater than or equal to 50 mg, very advantageously in an amount greater than or equal to 60 mg.

Alternatively, the composition comprises or consists of:
240 mg of vitamin C;
30 mg of vitamin E;
12.5 mg of zinc;
1 mg of copper;
10 mg of lutein;
2 mg of zeaxanthin;
950 mg of fish oil;
resveratrol, advantageously in an amount greater than or equal to 20 mg, very advantageously in an amount greater than or equal to 30 mg.

According to a particular embodiment, such a composition is devoid of at least one of the ingredients from the following list:
saffron;
a metal chelating agent, such as EDTA or phytic acid;
a nucleotide;
pycnogenol;
an extract of ginkgo biloba.

According to this embodiment, the composition used does not combine resveratrol with saffron, or resveratrol with a metal chelating agent and one or more nucleotides, or resveratrol and pycnogenol and/or an extract of ginkgo biloba.

A composition implemented within the scope of the invention may be present in the form of a liquid, solution, suspension, paste or gel.

Advantageously, it is in solid form, such as powder, tablets including effervescent tablets, pills, capsules or lozenges, prepared in a conventional manner using acceptable additives such as binding agents, filling agents, lubricants, disintegrating agents or wetting agents. The tablets may optionally be coated in a manner known to the person skilled in the art using sugars, films or enteric coatings.

Alternatively, other delivery systems may be used, such as preparations in soft gelatine or gelatine-based capsules, or formulations prepared using nanotechnology, such as nano-dispersions, nano-emulsions or nano-encapsulations.

Such a composition may further contain one or more additives such as colorants, pigments. or flavourings.

According to a particular embodiment, the composition is in the form of capsules, defining a given volume in which the composition is contained. The capsule breaks down in the digestive system in order to release the active ingredients and allow its assimilation by the body.

Within this context, conventional additives are:
glycerol, for example glycerol monostearate; and/or
soya oil; and/or
beeswax; and/or
soya lecithin.

Suitably, these additives are added to the composition owing to their ability to improve, for example, the flow properties or homogenisation thereof.

In addition, and in a manner known to the person skilled in the art, the capsule shell may be made from the following ingredients:
gelatine; and/or
red iron oxide; and/or
black iron oxide; and/or
sorbitol; and/or
glycerol.

In a manner known to the person skilled in the art, the distribution in this type of packaging is a compromise between:
a limited number of doses, in order to ensure proper compliance with the treatment;
a suitable volume of doses, allowing the formulation of all the ingredients and easy ingestion.

Advantageously, such a composition is administered daily, i.e., every day.

According to an advantageous embodiment, this composition is formulated as 2 capsules, each containing the ingredients listed above in half the quantity.

According to a first alternative, all of these ingredients are mixed and administered as a single composition. Alternatively, these ingredients may be combined in different manners to be formulated and/or administered simultaneously or staggered in time.

Characteristically, the composition used within the scope of the invention is administered orally, advantageously in the form of 2 daily doses. According to a particular embodiment, the composition is in the form of 2 capsules that can be taken with a small amount of water. They can be taken simultaneously, for example at the beginning of the main meal or during the main meal.

In view of the effect illustrated in the examples, such a composition is intended for administering to a subject receiving anti-VEGF treatment.

According to another aspect, an anti-VEGF treatment, as will be detailed below, is intended for administering to a subject taking a composition as defined above, advantageously orally, even more advantageously orally on a daily basis.

According to a particular embodiment and within the scope of the invention, an anti-VEGF treatment corresponds to the intake of an anti-VEGF agent, more precisely, an agent inhibiting the VEGF signalling pathway.

Advantageously, the anti-VEGF agent is selected from an antibody, an antibody fragment, a recombinant protein, a fusion protein and a mixture thereof.

According to a particular embodiment, the anti-VEGF agent is selected from the group consisting of: ranibizumab, bevacizumab, aflibercept, abicipar pegol, conbercept, faricimab, brolucizumab, their biosimilars and mixtures thereof.

In the frame of the invention, a biosimilar is understood, in the common sense of the term, to be a medicinal product similar to a biological substance produced from a living cell or organism, called the reference medicinal product. This biosimilar medicinal product must have similar physico-chemical and biological properties, the same pharmaceutical substance and the same pharmaceutical form as the reference medicinal product, and the equivalent efficacy and safety.

Ranibizumab is a humanised monoclonal antibody fragment against VEGF-A.

Bevacizumab is a recombinant humanised monoclonal antibody against VEGF.

Aflibercept is a recombinant fusion protein corresponding to the extracellular domains of human VEGF receptor types 1 and 2 (VEGF-R1 and VEGF-R2) fused to the crystallisable fragment (Fc) of human IgG1 immunoglobulin.

Abicipar pegol is an ankyrin repeat protein that binds to all VEGF-A isoforms. It is small in size (34 kDa) suggesting that it has a short intraocular half-life, but it acquires the pharmacokinetic characteristics of a larger protein notably a longer half-life via pegylation, i.e., the addition of polyethylene glycol molecules.

Conbercept is similar to aflibercept in that it involves a recombinant fusion protein corresponding to the key extracellular domains of human VEGF receptor types 1 and 2 and IgG Fc. However, conbercept also contains the fourth domain of the VEGF-2 receptor.

Faricimab is a bispecific anti-angiopoietin 2 and anti-VEGF monoclonal antibody.

Brolucizumab (or RTH258, Novartis) is a single-chain antibody that attains much higher antibody content in the eye than currently approved treatments. This antibody has a very high affinity for all VEGF-A isoforms and is expected to allow to space out the intravitreal injections.

Conventionally, these anti-VEGF agents are administered by intravitreal injection. However, in view of the potentiation of their effect in the presence of a composition as described above, their topical administration, advantageously at the eye surface, may be considered.

The combination of these two treatments (oral nutraceutical ophthalmic composition and anti-VEGF treatment by intravitreal injection or topically) is advantageous in relation to the treatment of eye diseases, advantageously retinal diseases with a neovascular component, even more advantageously selected from the group consisting of:
age-related macular degeneration, advantageously the wet form of AMD;
diabetic macular oedema (DMO);
retinal vein occlusion (RVO);
myopic choroidal neovascularisation (mCNV);
neovascular glaucoma (NVG); and
proliferative diabetic retinopathy (PDR).

According to another aspect, the invention relates to a combination product or combination preparation containing the nutraceutical composition and anti-VEGF treatment as described above for simultaneous, separate or sequential use in the treatment of an eye disease,

advantageously a retinal disease with a neovascular component, even more advantageously selected from the group consisting of:
age-related macular degeneration, advantageously the wet form of AMD;
diabetic macular oedema (DMO);
retinal vein occlusion (RVO);
myopic choroidal neovascularisation (mCNV);
neovascular glaucoma (NVG); and
proliferative diabetic retinopathy (PDR).

As already mentioned, and in this context, the nutraceutical composition is advantageously administered orally, even more advantageously on a daily basis, for example in the form of 2 capsules taken at the beginning or in the middle of the main meal.

On the other hand, anti-VEGF treatment is administered by intravitreal injection or topically. Intravitreal injection remains the preferred route of the administration, but current developments make it possible to consider topical administration, advantageously by application to the eye surface.

Indeed, as demonstrated, this combination makes it possible to reduce the dosage, notably of anti-VEGF treatment. Thus, it is possible to reduce the doses applied, or advantageously to space out the administrations (or reduce the frequency of the administration) which are typically spaced out by 1 to 3 months.

In other words, this combination product allows the dose and/or frequency of anti-VEGF treatment to be reduced in comparison with the dose and/or frequency applied to a subject not receiving the nutraceutical composition.

According to another aspect, the interval between two anti-VEGF treatments may be greater than 30 days, advantageously greater than 45 days, preferably greater than 60 days or even 75 days, more advantageously greater than 90 days compared with the current dosage.

Thus, and according to another aspect, the interval between two anti-VEGF treatments is greater than 30 days, advantageously greater than 45 days, preferably greater than 60 days or even 75 days, more advantageously greater than 90 days.

It is thus possible to reduce the potential toxicity of anti-VEGF treatments, notably their side effects. The spacing of their administration, which requires the intervention of specialists, is also interesting from the economic point of view.

### EXAMPLE EMBODIMENTS OF THE INVENTION

The invention and the advantages resulting from it appear more clearly from the following figures and examples that are given to illustrate the invention and in non-limiting manner.

### DESCRIPTION OF THE FIGURES

FIGS. 1A and 1B compare the evolution of VEGF levels after administration of AVASTIN®, in incubated undifferentiated (pathological) retinal cells (FIG. 1A) in the presence (lower curve) and absence (upper curve) of formula A, or (FIG. 1B) in the presence (lower curve) and absence (upper curve) of formula B.

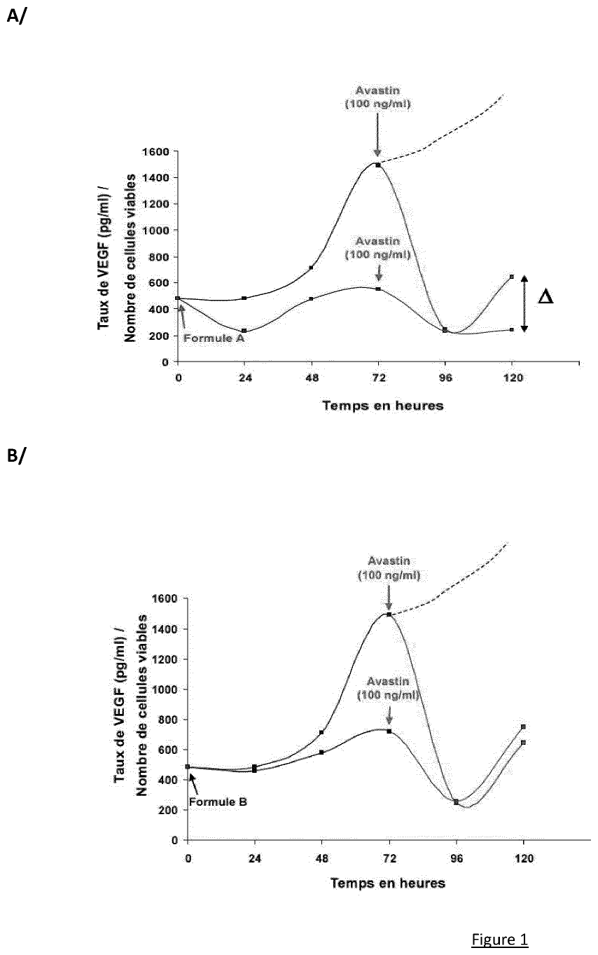

### A/PROTOCOL

### Cell Culture

In vitro ARPE-19 human retinal undifferentiated cells (ATCC® CRL-2302™) are cultured in DMEM (Dulbecco's modified eagle medium/nutrition mix F12 (1:1 Gibco, Paisley, UK) enriched with 10% foetal calf serum (FSS, Dominique DUTSCHER, South American origin, lot nºS15197S1810), 1% PSA (antibiotic cocktail: Penicillin, Streptomycin, and Amphotericin B respectively at 10,000 U/mL: 10 mg/mL: 25 μg/mL, PAN Biotech) and 15 mM HEPES (pH 7.4, Gibco). The cells are grown in an atmosphere at 37° C. and 5% CO₂. These cells may be assimilated to affected or pathological retinal cells.

### Treatments

Cells were grown and seeded in 24-well culture dishes at 60,000 cells per well in DMEM/F12 medium, 10% SVF and 1% PSA. 24 hours after seeding, the growth medium is removed and replaced with a restrictive DMEM/F12 growth medium with 1% SVF and 1% PSA without phenol red. After a further 24 hours, the cells are placed in the presence or absence of formula A (with resveratrol) or formula B (without resveratrol).

The composition of formulae A and B is described below:

TABLE 1
Composition of formula A (with resveratrol)

| | Quantities in | | Quantities in |
|---|---|---|---|
| Ingredients | mg/capsule | Content in mg/g | mg/day |
| Fish oil*, of which | 475 | 688 | 950 |
| DHA | 183 | 265 | 366 |
| EPA | 86 | 125 | 172 |
| Vitamin C | 60 | 87 | 120 |
| Vitamin E | 15 | 22 | 30 |
| Zinc | 6.25 | 9.1 | 12.5 |
| Copper | 0.5 | 0.7 | 1 |
| Lutein | 10 | 14.5 | 20 |
| Zeaxanthin | 2 | 2.9 | 4 |
| Resveratrol | 30 | 43 | 60 |

*Omegavie ® Oil TG Quality Silver Oil

Please note that one capsule contains 690 mg of the preparation, and that one daily dose of 2 capsules is recommended.

TABLE 2
Composition of formula B (without resveratrol)

| | Quantities in | | Quantities in |
|---|---|---|---|
| Ingredients | mg/capsule | Content in mg/g | mg/day |
| Fish oil*, of which | 512.3 | 674 | 512.3 |
| DHA | 250 | 329 | 250 |
| EPA (≥) | 56 | 74 | 56 |
| Vitamin B1 | 1.4 | 1.8 | 1.4 |
| Vitamin B2 | 1.6 | 2.1 | 1.6 |
| Vitamin B3 | 18 | 24 | 18 |
| Vitamin B6 | 2 | 3 | 2 |
| Vitamin B9 | 0.2 | 0.3 | 0.2 |
| Vitamin B12 | 0.001 | 0.001 | 0.001 |
| Vitamin C | 60 | 79 | 60 |
| Vitamin E | 10 | 13 | 10 |
| Zinc | 7.5 | 9.9 | 7.5 |
| Manganese | 1 | 1 | 1 |
| Selenium | 0.025 | 0.03 | 0.025 |
| Copper | 1 | 1 | 1 |
| Lutein | 10 | 13 | 10 |
| Zeaxanthin | 2 | 2.6 | 2 |
| Glutathione | 1 | 1 | 1 |

*Oil TG

Note that one capsule contains 760 mg of the preparation and that 1 capsule only is recommended to be taken per day.

Formulae A and B are taken at the mass of 0.115 g in 1 mL DMSO (dimethyl sulfoxide), so as to obtain a resveratrol equivalence of 10 mM for formula A and 0 mM for formula B. The contents of fish oil, vitamins C and E, Zinc, Copper, Lutein and Zeaxanthin are considered equivalent in the 2 preparations. From this solution, dilutions are made directly in the DMEM/F12 growth medium 1% SVF, 1% PSA without phenol red in order to reach contents of 10 μM and 20 μM necessary for the experiment.

After 24 hours of pre-incubation, the cells are treated with AVASTIN® 100 ng/ml (±formula A or B).

### ELISA (Enzyme-linked Immunosorbent Assay)

At the time of treatment (t0) with Avastin® 100 ng/ml as well as 24 hours, 48 hours, and 72 hours after treatment, the supernatant of the cells is collected to analyse the amount of VEGF-A released. Each ELISA is performed according to the precise instructions from the manufacturer (Human VEGF-A ELISA Kit BMS277 affymetrix eBioscience).

### Cristal Violet

The cell layer is analysed by crystal violet to determine cell viability. A ratio between the VEGF-A level from the supernatant and the corresponding cell layer can be performed.

The cell layer is gently rinsed twice with 1X PBS buffer. When the excess liquid is removed, the cell layer is fixed to its support by absolute ethanol. After an incubation time of at least 30 minutes, the absolute ethanol is removed. The cells are contacted with the crystal violet staining solution for 20 minutes at room temperature and under oscillation (25 oscillations per minute).

The crystal violet solution is removed, and the wells are rinsed 4 times with water in order to remove excess stain. The plates are then dried at room temperature (for 16 to 24 hours). The cell stain is then resuspended in a 33% acetic acid solution, the plates are put under oscillation and the optical density can be read by spectrometry at a wavelength of 570 nm (OD₅₇₀).

### B/Results

It can be observed that the presence of formula A makes it possible to limit the amount of VEGF produced, but it also significantly delays the rise of this level after treatment with an anti-VEGF, which would allow to space out the anti-VEGF injections.

Formula B, on the other hand, leads to a reduction in the amount of VEGF produced, but does not lead to a delay in the recovery of VEFG production in retinal cells treated with anti-VEGF.

This unexpected difference is related to the presence of an high content of resveratrol in formula A.

1-15. (canceled)

16. A method of treating an eye disease in a subject receiving an anti-VEGF treatment, comprising administering to the subject an ophthalmic nutraceutical composition comprising resveratrol.

17. The method of claim 16, wherein the composition comprises resveratrol in an amount of greater than 20 mg.

18. The method of claim 16, wherein the composition comprises resveratrol in an amount of 30 mg or greater.

19. The method of claim 16, wherein the composition comprises resveratrol in an amount of 50 mg or greater.

20. The method of claim 16, wherein the composition further comprises:
vitamins;
trace elements;
carotenoids; and
omega-3 fatty acids.

21. The method of claim 20, wherein the vitamins comprise vitamin C or vitamin E.

22. The method of claim 20, wherein the trace elements comprise zinc or copper.

23. The method of claim 20, wherein the carotenoids comprise zeaxanthin or lutein.

24. The method of claim 20, wherein the omega-3 fatty acids are from fish oil.

25. The method of claim 20, wherein the omega-3 fatty acids comprise eicosapentaenoic acid (EPA) or docosahexaenoic acid (DHA).

26. The method of claim 16, wherein the composition comprises:
vitamin C present in an amount of 50 mg to 500 mg;
vitamin E present in an amount of 10 mg to 500 mg;
zinc present in an amount of 5 mg to 100 mg;
copper present in an amount of 0.2 mg to 10 mg;
lutein present in an amount of 2 mg to 50 mg;
zeaxanthin present in an amount of 0.5 mg to 10 mg; and
omega-3 fatty acids from fish oil present in an amount of 100 mg to 1000 mg.

27. The method of claim 26, wherein the composition comprises:
vitamin C present in an amount of 120 mg;
vitamin E present in an amount of 30 mg
zinc present in an amount of 12.5 mg;
copper present in an amount of 1 mg;
lutein present in an amount of 20 mg;
zeaxanthin present in an amount of 4 mg; and
omega-3 fatty acids from fish oil present in an amount of 800 mg to 1000 mg.

28. The method of claim 16, wherein the composition is administered orally.

29. The method of claim 16, wherein the composition is administered daily.

30. The method of claim 16, wherein the anti-VEGF treatment comprises administration of an inhibitor of the VEGF signaling pathway.

31. The method of claim 30, wherein the inhibitor comprises an antibody, an antibody fragment, a recombinant protein, a fusion protein, or a mixture thereof.

32. The method of claim 30, wherein the inhibitor comprises ranibizumab, bevacizumab, aflibercept, abicipar pegol, conbercept, faricimab, brolucizumab, or a mixture thereof.

33. The method of claim 16, wherein the anti-VEGF treatment is administered by intravitreal injection or topically.

34. The method of claim 16, wherein the eye disease is selected from the group consisting of age-related macular degeneration, diabetic macular oedema, retinal vein occlusion, myopic choroidal neovascularisation, neovascular glaucoma, and proliferative diabetic retinopathy.

35. The method of claim 16, wherein the composition is administered simultaneously, separately, or sequentially with the anti-VEGF treatment.

36. The method of claim 16, wherein the dose and/or frequency of the anti-VEGF treatment is reduced in comparison with the dose and/or frequency applied to a subject who is not receiving the composition.

37. The method of claim 36, wherein the frequency between two administrations of the anti-VEGF treatment is reduced by greater than 30 days compared with the frequency between two administrations of the anti-VEGF treatment in a subject not receiving the composition.
